(19) United States
(12) Patent Application Publication
(10) Pub. No.: US 2010/0298692 A1
(43) Pub. Date: Nov. 25, 2010

(54) METHOD FOR DETECTING TUMOR CELL INVASION USING SHORT DIFFUSION TIMES

(75) Inventors: Kathleen Schmainda, Elm Grove, WI (US); Eric S. Paulson, Wauwatosa, WI (US); Douglas E. Prah, Muskego, WI (US)

Correspondence Address: POLSINELLI SHUGHART PC, 700 West 47th Street, Suite 1000, KANSAS CITY, MO, 64112, US

(73) Assignee: IMAGING BIOMETRICS, LLC, Elm Grove, WI (US)

(21) Appl. No.: 12/601,247

(22) PCT Filed: May 22, 2008
(86) PCT No.: PCT/US08/64599

§ 371 Date: May 24, 2010

Related U.S. Application Data
(60) Provisional application No. 60/939,546, filed on May 22, 2007.

(51) Int. Cl. A61B 5/055

(52) U.S. Cl. 600/410

(57) ABSTRACT

An improved method for detecting tumor cell invasion using diffusion times as short as two (2) msec incorporates diffusion weighing imaging techniques into a standard spin echo (SE) pulse sequence to minimize the effects of compartment boundary restrictions on diffusion values and corresponding MRI imaging data related to glioma invasion.

### CROSS-REFERENCE TO RELATED APPLICATIONS

This application is the National Stage of International Application No. PCT/US2008/064599 filed May 22, 2008, which claims the benefit of U.S. Provisional Patent Application Ser. No. 60/939,546 filed on May 22, 2007, the entire contents of each of which are incorporated herein by reference.

### BACKGROUND OF THE INVENTION

Remedial surgical and radiological techniques and procedures for brain tumors require precise knowledge of the boundary of the tumor cell mass. Standard magnetic resonance imaging (MRI) techniques do not adequately reflect the cellular and microscopic structure of the tumor, particularly areas of tumor cell invasion. Magnetic resonance imaging relies on the relaxation properties of excited hydrogen nuclei in water and in lipids. Water molecules are in constant motion, and the rate of movement or diffusion is temperature dependant and also depends upon the kinetic energy of the molecules. However, in biological tissues, diffusion is not truly random because tissue has structure which limits or restricts the amount of diffusion possible. Moreover, chemical interactions of water and the macromolecules which may be contained in the water also may affect diffusion properties.

Diffusion weighted imaging (DWI), is one approach used to document tumor tissue structure. DWI produces in vivo images of biological tissues weighted with characteristics of water diffusion across local microstructures. To obtain diffusion-weighted images, a pair of strong gradient pulses is added to a standard spin echo (SE) pulse sequence. The first pulse dephases the spins, and the second pulse rephases the spins if no net movement occurs. If net movement of spins occurs between the gradient pulses, signal attenuation or suppression results. The more diffusion that occurs at a given location, the more attenuated (less intense) is the image at a given location.

The length of the diffusion experiment is very important, inasmuch as the result is dependant upon the time over which diffusion is measured. The selected diffusion time determines the degree to which the protons “survey” the microscopic structures. Present state of the art techniques limit DWI in clinical practice to diffusion times greater than approximately 20 msec. At such long diffusion times, most tissue water will experience a boundary or other restriction such as a cell membrane, which will result in a plateauing of diffusion values such that the diffusion from all compartments will begin to look the same. At shorter diffusion times, diffusion becomes more sensitive to the intracellular environment and thus more sensitive to the changes that can occur in the cell as a result of tumor cell invasion. Thus there exists a need for an improved MRI technique using short diffusion times to detect tumor cell invasion.

### SUMMARY OF THE INVENTION

The present invention discloses an improved method for detecting the presence of tumor cell invasion which reduces diffusion times to as little as 2 msec. by incorporating isotropic diffusion weighing into a standard spin echo (SE) pulse sequence using a pair of balanced bipolar gradients positioned around a refocusing pulse. The DWI protocol is optimized to be sensitive to the presence of invading tumor cells, and a new algorithm is employed post-imaging to analyze the data. The data may be fit to a stretched exponential model, a monoexponential model, a biexponential model or a kurtosis model.

### BRIEF DESCRIPTION OF THE DRAWINGS

FIG. 1 depicts a normalized diffusion weighted signal with a diffusion time of 2.33 msec.;

FIG. 2 illustrates a bipolar spin echo planar diffusion weighted sequence using one pair of bipolar lobes;

FIG. 3 illustrates a bipolar spin echo planar diffusion weighted sequence using four pair of bipolar lobes;

FIGS. 4(a) and (b) illustrate diffusion weighted signals obtained from an ethanol phantom;

FIG. 5 depicts gradient waveforms obtained from a hall probe recording the gradient for sixteen pair bipolar spin echo diffusion weighted sequences;

FIGS. 6(a) and (b) illustrate calculated apparent diffusion coefficients for ethanol and water as a function of exchange times;

FIGS. 7(a)-(c) illustrate ADC maps under various sequences;

FIG. 8 depicts hemtoxylin stained sections from a tumor-bearing rat; and

FIG. 9 illustrates alpha maps from the stretched exponential fit of a representative C6 tumor-bearing rat.

### DESCRIPTION OF THE PREFERRED EMBODIMENT

Current DWI techniques implemented with relatively long diffusion times have a reduced sensitivity to intracellular compartments, such as the nuclear compartment, which is known by histomorphometric techniques to be altered. The detection method of the present invention is designed to expose the intracompartmental signals that are overlooked by DW sequences using longer diffusion times and to provide new information useful for glioma localization.

A Simulation of the Effects of Compartment Size on the Diffusion Weighted Signal.

Diffusion weighted MR obtains estimates of the diffusion coefficient by allowing ensembles of spins to course through the medium over a known time. For a given signal attenuation the diffusion coefficient can be calculated from the length of the diffusion experiment and the gradient shape. In vivo this estimate is influenced by restrictive boundaries and other objects that hinder the ensemble's translational motion. If the time given for the ensemble of spins to migrate is short enough, the effects of restriction will be reduced. As the length of the diffusion experiment increases, spins will have more time to interact with the microstructure, further impeding the translational motion of the spins. Consequently, the calculated apparent diffusion coefficient (ADC) strongly depends on diffusion time and will approach an asymptotic value as the length of the diffusion experiment increases.

The effects of a compartment volume on a diffusion-weighted signal may be numerically demonstrated using a Matlab simulation. For purposes of a simulation, the range of compartment sizes was chosen such that they reflected the actual range of in vivo intracellular compartment diameters. Ensembles of protons were uniformly distributed within impermeable spheres of radius, R. For every time step, each proton's new position was normally distributed with mean was equal to the mean radial displacement calculated using the Einstein Relation, <x>=(6Dτ)½, and assuming that the medium was very homogeneous, the standard deviation of the of the radial displacement was assigned a value of one tenth the mean displacement. A diffusion coefficient of 2.0E-3 mm2/sec was assumed for the calculation of the mean and standard deviation of the translation step. The position of a proton that traversed the radius of the sphere was recalculated until it fell within the sphere. Accumulated phase and corresponding signal attenuation was calculated for each sphere using a bipolar SE DW sequence (one pair of bipolar lobes, one bipolar lobe on each side of the 180° refocusing pulse) with a constant diffusion time of 2.66 msec.

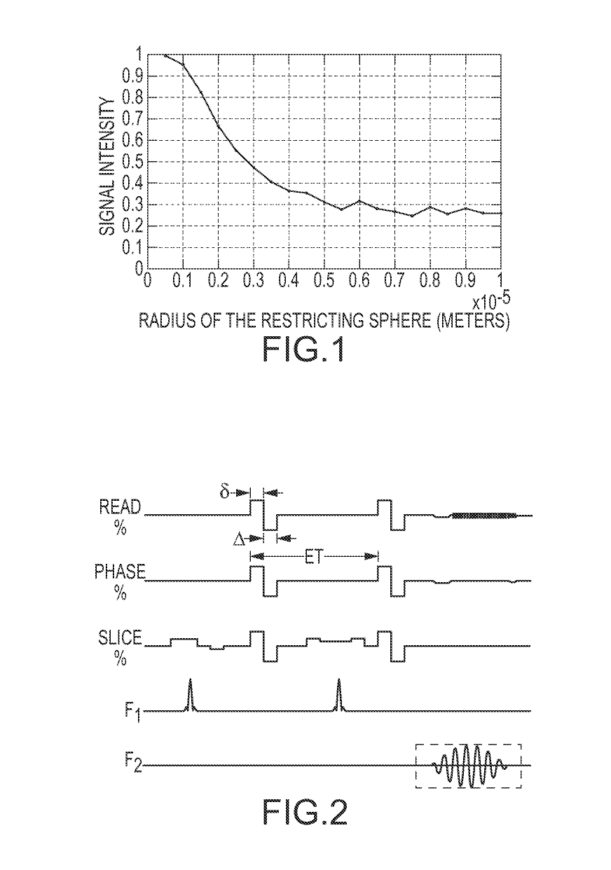

The simulation results, shown in FIG. 1, demonstrate that as the size of the compartment decreases the diffusion weighted (DW) signal is attenuated, thereby resulting in an underestimation of the actual unrestricted diffusion coefficient. As the size of the compartment increases, the number of spins interacting with the restricting boundary of the compartment decreases, reducing the effects of diffusion. Results from this simulation suggest a diffusion weighted sequence with a diffusion time long enough to allow most spins to interact with the restrictive boundaries will have an insignificantly contribution to the DW signal attenuation. This implies that smaller intracellular compartments, such as the mitochondria and nucleus, exhibit a decreased influence on the DW signal. Increased intracellular sensitivity to the nucleus and mitochondria, which histomorphologic evidence has verified to be altered in gliomas cells, will be achieved if shorter diffusion times (t<20 msec) are used.

The compartmentalization of tissue water is known in the art, and two dimensional single cell images reveal that the nucleus and cytoplasm represent two separate compartments with distinct diffusion coefficients. In vivo rat studies using cesium-133, an MR active potassium mimetic that primarily resides in the intracellular space have identified three chemical shift imaging (CSI) peaks. The ratio of the area of the small peak to the two larger peaks agrees with published ratios of intracellular to extracellular space. Furthermore, diffusion weighted chemical shift imaging found that each peak exhibited a unique diffusion weighted signal attenuation, all of which strongly suggest that two distinct compartments with unique diffusion related properties exist within the intracellular compartment.

Implementation of the Short Time Diffusion Weighted Sequence.

In the past, hardware limitations have restricted the maximum gradient strength making it impossible to achieve short diffusion times with the widely used pulsed gradient spin echo experiment. Clinical limitations on gradient switching rates also exist. Accordingly, studies of short diffusion times in vivo have been limited. Fortunately, other methods of diffusion weighting which can circumvent these limitations are feasible. For studies conducted on anesthetized animal models, where gradient switching rates are not limited, many rectangular bipolar lobes can be used to obtain higher diffusion weighting. Due to the ease of implementation and higher achievable diffusion weighting, rectangular bipolar lobes were chosen as the short time diffusion weighted sequence used. For implementation on systems with weaker gradient strength, other alternatives are possible, by way of example, sinusoidal or oscillating gradients may be used to achieve diffusion weighting. Sinusoidal gradients can achieve stronger diffusion weighting without rapid gradient switching rates above the Food and Drug Administration (FDA) limits.

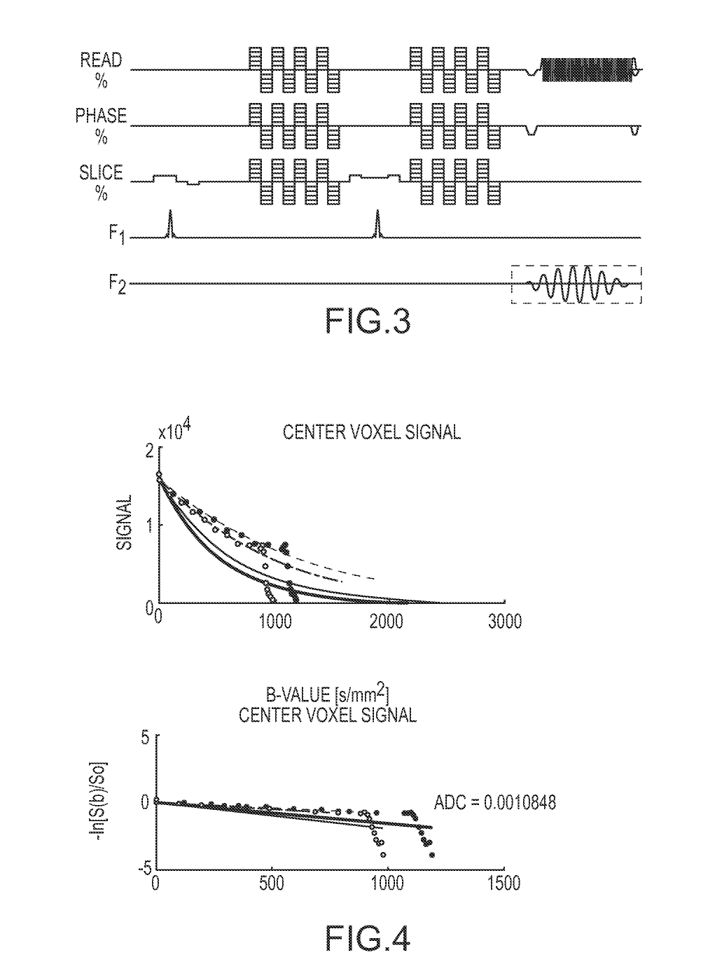

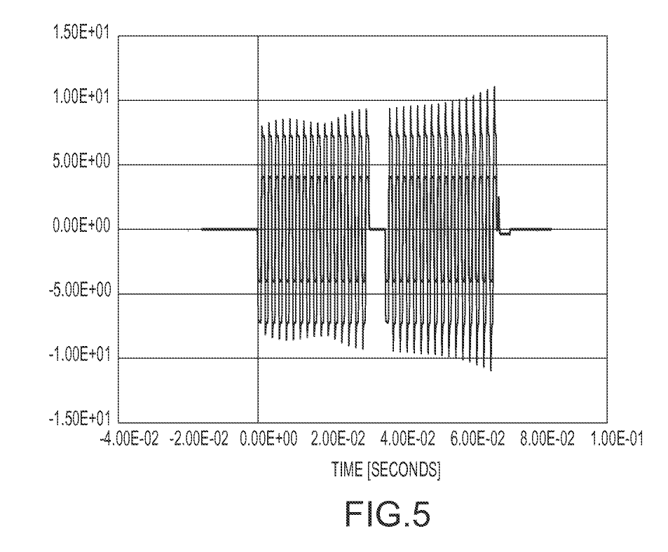

In development of the methodology which is the subject of the present invention, short diffusion times were achieved by incorporating isotropic diffusion weighting into a standard spin echo (SE) pulse sequence using a pair of balanced bipolar gradients positioned around the refocusing pulse. These modifications were also incorporated into a SE echo planar sequence. The incorporation of bipolar SE diffusion weighting with one pair of bipolar gradients (one balanced bipolar waveform for each gradient on each side of the 180° refocusing pulse) into the SE echo planar sequence is depicted in FIG. 3. A bipolar SE diffusion weighting sequence with four pair of bipolar lobes (four balanced bipolar waveforms for each gradient on each side of the 180° refocusing pulse) is depicted in FIG. 4. The same notation commonly used to describe both monopolar and bipolar diffusion pulses was chosen with the necessary addition of a separation parameter. The sequence was implemented with up to 16 pair of bipolar pulses. The diffusion weighting can be calculated as follows:

$${b}_{BSE} = \frac{4}{3} n {γ}^{2} {\vec{G}}^{2} {δ}^{3}$$

where n is the number of pair of bipolar diffusion weighting lobes. Diffusion exchange weighted (DEW) imaging techniques demonstrate that sequences using two separate diffusion experiments preformed in the same sequence separated by a known time, exchange time, ET, can be used to determine the compartmental exchange properties of the diffusing spins. In summary, the sequence is not only capable of achieve diffusion times as short as 1 msec with the use of the currently available gradient strengths, but also provides the potential of measuring underlying compartmental exchanges properties.

Validation of the Accuracy of the Diffusion Weighted Sequence.

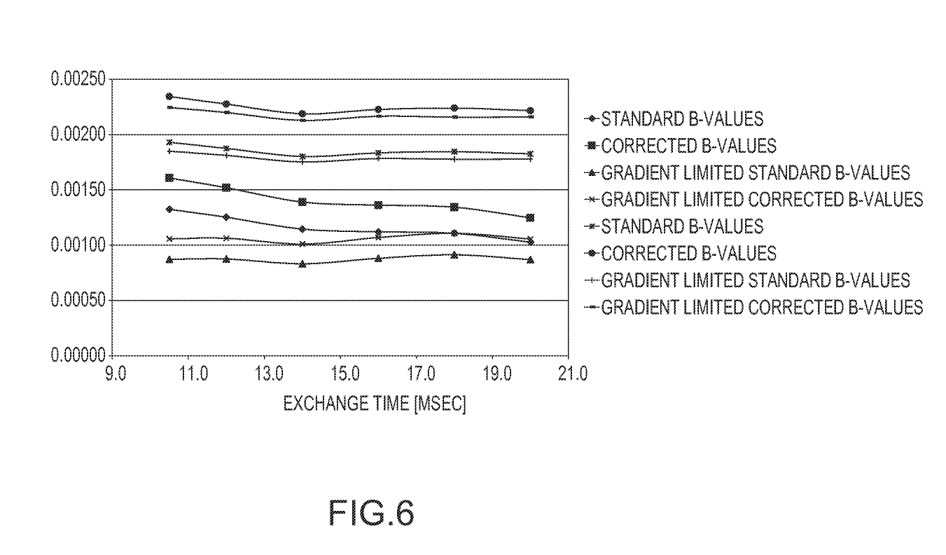

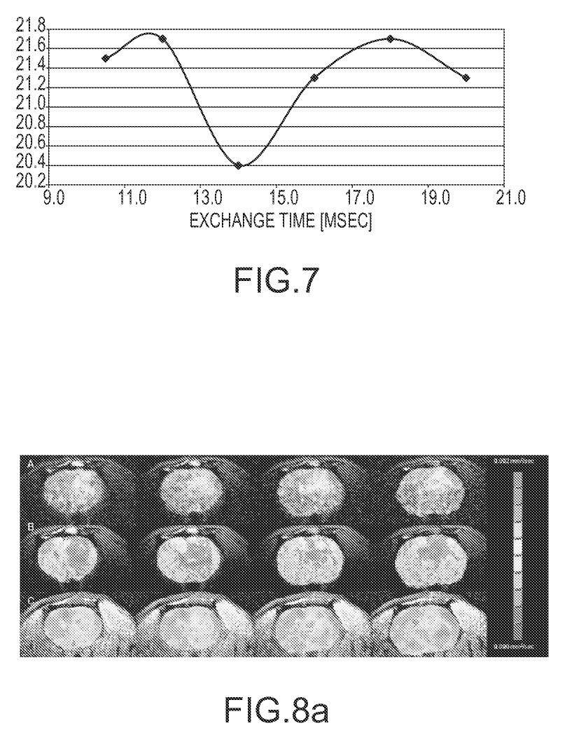

All studies were performed on a Bruker Biospec 30 cm 9.4 T using local gradient coils capable of achieving maximum gradient strengths of 40 G/cm per channel. Validation of the accuracy of the DW sequence was preformed in multiple stages. Before validation, preliminary phantom data reveal that the b-values were incorrectly calculated. FIG. 5 displays raw data collected from the ethanol phantom. It was determined that the gradient amplifiers were not able to consistently generate balanced waveforms at strengths greater than 85% of maximum. FIG. 6 displays the output from Hall probes designed to measure the actual gradient current. The blue curve is an example of the bipolar DW SE sequence using a gradient strength greater than 85% maximum and the red curve less than 85%. The pre-emphasis gradients on the blue curve are changing from pulse to pulse, which results in unbalanced bipolar lobes. All subsequent experiments were gradient limited to 85% maximum. Furthermore, it was determined that due to the short pulse widths used the actually shape of the gradient waveform had to be taken into account when calculating b-values. FIG. 5 displays raw data (dark blue points), b-value corrected data (green points), the least square fit of the raw data (solid green line), the least square fit of the gradient limited raw data (dashed green line), the least square fit of the b-value corrected raw data (solid red line), and the least square fit of the b-value corrected gradient limited raw data (dashed red line). To ensure that bipolar diffusion weighting gradients were correctly balanced experiments using water and ethanol phantoms were performed. Any residual phase accumulation due to unbalanced gradients would, as the ET increased, decrease the DW signal and overestimate the ADC. Results shown in FIG. 7 show a no such relationship with the calculated ADC (gradient-limited corrected b-values). The slight variation in ADC for both ethanol and water are explained by corresponding fluctuations in observed temperature. The calculated ADC for ethanol and water were in agreement with published values over the recorded temperature range for the variable exchange experiments.

After determining that the bipolar gradients were balanced, diffusion coefficients were calculated for a various number of diffusion lobes of different widths, δ. The ADCs were again calculated from 20 b-values ranging from 0 to 1760 s/mm2. FIG. 6 displays the ADCs from the ethanol and water phantom. For gradient widths of 1 msec and greater the ADC were correctly calculated. Unfortunately, the gradient rise time did not allow for a correct estimation of the ADC using a gradient width of 0.5 msec. The number of diffusion lobes was also not found to impact the estimated ADC. The effects of eddy currents were assumed negligible after measurements of ethanol and water phantoms revealed no parameter dependent significant differences in the calculated diffusion coefficient from literature values. Diffusion times, gradient amplitudes, and separation of the bipolar lobes were each independently varied.

Preliminary Rat Data.

Twelve male Sprague-Dawley rats were inoculated, intracerebrally, with 10̂5 (10 μL) C6 glioma cells and imaged 14 days post-inoculation. Rectal temperature was monitored and maintained at 37° C.±1° C. Three series of DW images were collected. For Set A, a standard clinical SE DW images were acquired with TE=59.1 msec, TR=2 sec, δ=25 msec, Δ=30 msec, τ≈22 msec, b-value=1000 s/mm2. For Set B, a set of bipolar SE DW images were acquired with TE=49.1 msec, TR=2 sec, Δ=δ=10 msec, τ≈6.66 msec, 20 b-values ranging from 0 to 1760 s/mm2. For Set C, a set of bipolar SW DW images were acquired with a shorter TE=25.1 msec, TR=2 sec, Δ=δ=4 msec, τ≈2.66 msec, 20 b-value ranging from 0 to 1760 s/mm2. Images were acquired with a round single turn RF coil. Immediately following imaging rats were sacrificed, brains were extracted, sectioned into 7 μm thick slices, and stained with hematoxylin.

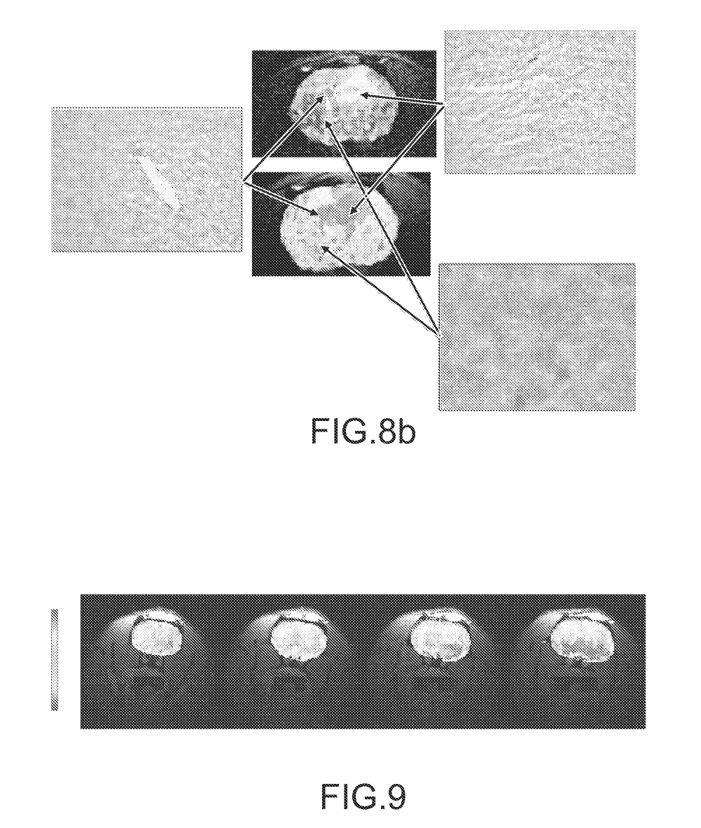

Apparent diffusion coefficient (ADC) maps overlayed on T2 weighted (b=0 s/mm2) images from one representative rat are shown in FIG. 8. The tumor inoculation site can be seen above the right side of the image. The first row shows ADC maps collected with standard clinical imaging parameters (i.e. b=1000, τ=22 ms). Hemtoxylin stained sections, data shown in FIG. 9, suggests a much larger area of tumor invasion than depicted in Row A. The second and third rows show ADC maps obtained using diffusion times of 6.66 and 2.66 msec, respectively. Thus, the spatial distribution and reported ADC values depend profoundly on the choice of τ and TE.

The DW images had different T2 weighting due to the differences in echo times. The nucleus has a longer T2 and a larger diffusion coefficient than the cytoplasm. Furthermore, proliferating tumor cells have increased nuclear to cytoplasmic ratios, thus suggesting longer intracellular diffusion times and T2s of proliferating tumor cells. These characteristic diffusion and T2 values may explain the results shown in FIG. 8. The results in row B obtained with τ=6.66 ms/TE=59.1 ms show larger areas of high ADC compared to the results shown in Row A, obtained for longer τ=22 ms, and the same TE (59.1 ms). For the results in row C, at shorter τ (2.66 ms) and shorter TE (25.1 ms) the area of high ADC is again different. In this case the shorter TE may be allowing for a more equal contribution from all compartments, both short and long TE compartments, to the diffusion signal. Thus this latter result may be most representative of the true distribution of invading tumor cells. The results of this study demonstrate an important dependence of diffusion results on the choice of the diffusion experimental parameters.

Application of the Stretched Exponential Model to Constant Gradient Diffusion Data.

As described above, diffusion times greater than at least 20 msec appear to be independent of diffusion time. Prior art in vivo rat studies utilizing diffusion times shorter than 20 msec reveal substantial differences between the methods of collecting DWI data. One such study compared DWI collected with constant diffusion times (Ct), constant diffusion gradients (Cg), and constant b-value (Cb), while varying the other parameters, found a marked difference, presumably due the effects of restriction, on diffusion measurement. [Niendorf, T., D. G. Norris, and D. Leibfritz, Detection of apparent restricted diffusion in healthy rat brain at short diffusion times. Magn Reson Med, 1994. 32(5): p. 672-7]. Diffusion times down to 1.6 msec were achieved using a single bipolar pulse. Significant differences were seen in the striatum and cortex between the Ct and Cg experiments. The Ct experiment produced monoexponential signal decay while the Cg produced non-monoexponential signal decay. Unlike most studies identifying multi-exponential signal attenuation which use a large range of b-values, this study found non-exponential attenuation only when varying the diffusion time but while holding the diffusion time constant. Consequently, a marker capable of quantifying the degree of microscopic restriction, presumably revealed due to the changes in diffusion time creating the non-monoexponential signal attenuation, would be valuable and potentially reveal information about the underlying compartment's geometry.

The stretched exponential model has shown promise as a marker to identify microscopic heterogeneity. If applied to the Cg experiment, alpha will reflect the effects that diffusion time have on the DW signal attenuation and therefore the degree of restriction. Preliminary data was collected on 6 Sprague Dawley rats with a diffusion time ranging from 1 to 6.66 msec using the same methods described above with respect to preliminary rat data. The stretched exponential model was then fit to the Cg data. The observed non-monoexponenital behavior was not as dramatic as was seen by Niendorf et al above. However, in that study the diffusion time had a broad rate of diffusion times, 1.6 to 11 msec, which would further contribute to the non-monoexponential behavior. A representative data set from one rat is displayed in FIG. 10. It is apparent that restriction across the rat brain is not uniform. However, the effect of restriction my not be completely seen for such a broad range of diffusion times.

Changes may be made in the above methodology without departing from the scope hereof. It should thus be noted that the matter contained in the above description and/or shown in the accompany figures should be interpreted as illustrative and not in a limiting sense. The following claims are intended to cover all generic and specific features described herein, as well as all statements of the scope of the present invention which, as a matter of language, might be said to fall therebetween.

What is claimed is:

1. A method for producing an image of an organ or tissue using a Magnetic Resonance Imaging (MRI) system comprising the steps of:
a) acquiring diffusion-weighted images using the MRI system, wherein diffusion-weighted images are acquired, in at least one direction, for a range of diffusion-weightings having at least two b-values using short diffusion times;
b) computing image maps of apparent diffusion coefficient (ADC); and
c) fitting the data to a stretched exponential model to create maps of an index of tissue heterogeneity (a) and the distributed diffusion coefficient (DDC).

2. The method of claim 1 wherein the short diffusion times of step a) are less than 25 msec.

3. The method of claim 1 wherein step b) is performed by computing the ADC, on a per-voxel basis, from MRI signals collected at a minimum of two different b-values.

4. The method of claim 1 wherein step c) is performed by:
a) computing the stretching parameter α, on a per-voxel basis, by fitting to the stretched-exponential model the diffusion-weighted MRI signals as a function of b-value, and
b) computing the stretching parameter DDC, on a per-voxel basis, by fitting to the stretched-exponential model the diffusion-weighted MRI signals as a function of b-value.

5. The method of claim 1 in which the pulse sequence is a single or multi-shot spin-echo echo planar imaging (SE-EPI) sequence with one or more pairs of balanced shaped bipolar gradients positioned around the refocusing pulse.

6. The method of claim 1 in which the pulse sequence is a single or multi-shot spin-echo spiral-based sequence with one or more pairs of balanced shaped bipolar gradients positioned around the refocusing pulse.

7. The method of claim 1 in which the diffusion weightings are either oscillating or sinusoidal.

8. The method of claim 1 wherein step a) includes the use of parallel imaging methods to acquire the diffusion-weighted images.

9. The method as recited in claim 1 wherein step a) includes the use of three dimension spiral-based imaging methods to acquire the diffusion-weighted images.

10. A method for producing an image of an organ or tissue using a Magnetic Resonance Imaging (MRI) system comprising the steps of:
a) acquiring diffusion-tensor images (DTI) using the MRI system, wherein diffusion-weighted images are acquired in at least 6 different directions for a range of diffusion-weightings having at least two b-values and using short diffusion times;
b) computing image maps of apparent diffusion coefficient (ADC), fractional anisotropy (FA), and the diffusion tensor; and
c) fitting the data to a stretched exponential model to create maps of an index of tissue heterogeneity, α, and the distributed diffusion coefficient (DDC).

11. The method of claim 10 in which step b) is performed by computing ADC, FA or DTI images from MRI signals collected at a minimum of two different b-values.

12. The method of claim 10 in which step c) is performed by:
a) computing the stretching parameter α, on a per-voxel basis, by fitting to the stretched-exponential model the diffusion-weighted MRI signals as a function of b-value; and
b) computing the stretching parameter DDC, on a per-voxel basis, by fitting to the stretched-exponential model the diffusion-weighted MRI signals as a function of b-value.

13. The method of claim 10 in which the pulse sequence is a single or multi-shot spin-echo echo planar imaging (SE-EPI) sequence with one or more pairs of balanced bipolar gradients positioned around the refocusing pulse.

14. The method of claim 10 in which the pulse sequence is a single or multi-shot spin-echo spiral-based sequence with one or more pairs of balanced shaped bipolar gradients positioned around the refocusing pulse.

15. The method of claim 10 in which the diffusion weighting are either oscillating or sinusoidal.

16. The method of claim 10 wherein step a) includes the used of parallel imaging methods to acquire the diffusion-weighted images.

17. The method of claim 10 wherein step a) includes the use of three dimensional spiral-based imaging methods to acquire the diffusion-weighted images.

18. The method of claim 1 wherein the short diffusion times are less that 25 msec.

19. The method of claim 10 wherein the short diffusion times are less that 25 msec.
